(19) United States
(12) Patent Application Publication
(10) Pub. No.: US 2012/0088847 A1
(43) Pub. Date: Apr. 12, 2012

(54) Synthetic Thickeners for Cosmetics

(75) Inventors: Gianmarco Polotti, Sesto San Giovanni (IT); Arianna Benetti, Gallarate (IT); Franco Federici, Busto Arsizio (IT); Giuseppe Li Bassi, Gavirate (IT)

(73) Assignee: Lamberti USA, Inc., Albizzate (IT)

(21) Appl. No.: 13/252,087

(22) Filed: Oct. 3, 2011

Related U.S. Application Data
(63) Continuation-in-part of application No. 10/541,565, filed on May 31, 2006, now abandoned.

(30) Foreign Application Priority Data
Jan. 9, 2003 (IT) VA2003A000002
Dec. 28, 2003 (EP) PCT/EP03/51056

(51) Int. Cl. A61K 8/92; A61Q 19/00

(52) U.S. Cl. 514/772.6

(57) ABSTRACT

Disclosed are inverse emulsions useful as thickeners for cosmetic formulations wherein the weight ratio between the aqueous phase and the oil phase is from 4:1 to 2:1 and containing from 20 to 70% by weight of an anionic acrylic polymer obtained by inverse emulsion polymerization of one or more anionic acrylic monomers, at least one of which containing a strongly acidic functional group, dissolved in the aqueous phase, and at least a hydrophobic acrylic monomer dissolved in the oil phase before the mixing of the two phases. Also disclosed is the procedure for their preparation.

### CROSS REFERENCE TO RELATED APPLICATIONS

This application is a continuation in part of the U.S. patent application having the Ser. No. 10/541,565 which was filed on May 31, 2006, which was a Rule 371 filing of PCT/EP2003/51056 with an International Filing Date of Dec. 28, 2003 and claiming priority from Italian Application Ser. No. VA2003A000002 filed Jan. 9, 2003.

### TECHNICAL FIELD

The present invention relates to inverse emulsions useful as thickeners in cosmetic formulations and to the Procedure for their preparation. Cosmetic formulations include all the products normally used for personal care, such as body and face creams, cleansing fluids, after-shave balms, foundation creams and other products for similar applications.

### BACKGROUND ART

It is known that a technical problem often encountered in the cosmetic industry is to obtain high viscous formulations (pastes, gels) which are stable over time.

An essential characteristic of the thickeners employed in cosmetic formulations is that they manifest their thickening capability even when used in small quantities, without negatively altering the other properties of the formulations.

In the specialized literature many methods are reported to regulate the rheological properties of different formulations, often including the use of polymers in the form of inverse emulsion (an inverse emulsion is an emulsion containing both an oil-in-water emulsifier and a water-in-oil emulsifier, wherein the aqueous phase is dispersed in the organic phase in very small drops).

We cite, as an example, EP 503853, wherein an inverse emulsion containing a polymer comprising units deriving from acrylamide, 2-acrylamido-2-methylpropanesulfonic acid and a polyfunctional monomer is described.

A disadvantage of the inverse emulsions of EP 503853 is the fact that they may contain traces of acrylamide monomer, a toxic substance which is unacceptable by the present European legislative trend.

In U.S. Pat. No. 6,375, 959 and U.S. Pat. No. 6,197, 287, which are both incorporated herein by reference in their entirety, a procedure for the preparation of cross-linked or branched anionic polyelectrolytes based on strongly acidic monomers and other monomers (but not acrylamide nor hydrophobic monomers), in the form of an inverse emulsion, is described.

The lack of stability of the emulsions used as thickeners in cosmetics, even if it is not a determining characteristic in view of the final properties of the finished cosmetic product itself, may cause troubles during their preparation, storing and transport.

It is highly desirable in the cosmetic field to have thickeners in the form of emulsion that, besides conferring a perfect homogeneity and showing both good thickening efficiency in different conditions and ease of use, are commercially available as stable emulsions and are able to give stable cosmetic formulations.

With the expression “stable emulsion” we mean an emulsion that in the normal storing conditions (from −10° C. to 40° C.) and for the usual lifetime (180-360 days) does not show phase separation, sediment, formation of floating pellicles and lumps.

With the expression “stable cosmetic formulation” we mean a thickened cosmetic formulation that in the above said conditions and lifetime does not show phase separation, sediment, formation of floating pellicles and lumps.

### DISCLOSURE OF INVENTION

It has now surprisingly been found that the inverse emulsions containing an anionic acrylic polymer obtained by inverse emulsion polymerization of one or more anionic acrylic monomers, at least one of which containing a strongly acidic functional group, dissolved in the aqueous phase, and at least a hydrophobic acrylic monomer dissolved in the oil phase before the mixing of the two phases, possess a stability which is perfectly suited for their industrial use in cosmetic formulations, even many months after their preparation

In the present text with the expression “anionic acrylic monomers” we mean both acrylic monomers containing a strongly acidic functional group, at least some of which being in neutral salt form, and acrylic monomers containing a carboxylic group.

It is a fundamental object of the present invention an inverse emulsion for the preparation of cosmetic formulations wherein the weight ratio between the aqueous phase and the oil phase is from 4:1 to 2:1 and containing from 20 to 70% by weight of an anionic acrylic polymer obtained by inverse emulsion polymerization of one or more anionic acrylic monomers, at least one of which containing a strongly acidic functional group, dissolved in the aqueous phase, and at least a hydrophobic acrylic monomer dissolved in the oil phase before the mixing of the two phases, the percentage of the hydrophobic acrylic monomers on the total weight of the anionic acrylic monomers being of 0.1% to 5% by weight, preferably of 0.5 to 1.5% by weight.

It is a further object of the present invention a procedure for the preparation of an inverse emulsion for cosmetic formulations characterized by:
a. adding to a mixture of water and one or more anionic acrylic monomer, at least one of which containing a strongly acidic functional group, an aqueous solution of an alkali to regulate the pH between 4 and 10, a cross-linking agent and an initiator of radical polymerization, maintaining the temperature between 0 and 5 C;
b. preparing an oil phase containing from 0.1 to 10% by weight of at least one hydrophobic acrylic monomer and one or more water-in-oil emulsifiers;
c. introducing the mixture obtained in a. into the oil phase prepared in b. and emulsifying the two phases by vigorous stirring;
d. initiating the polymerization and completing it, maintaining the temperature between 55 and 95 C, under vigorous stirring; and
e. the anionic acrylic monomer containing a strongly acidic functional group is selected among the monomers of this kind that are normally employed for the preparation of polymeric synthetic thickeners for the cosmetic use, such as 2-acrylamido-2-methylpropanesulfonic acid and its salts.

In the present text with the expression “hydrophobic acrylic monomer” we mean an acrylic monomer which is insoluble in water.

For the realization of the present invention the preferred hydrophobic acrylic monomers are esters of acrylic or methacrylic acid with C₄-C₂₀ linear or branched monofunctional alcohols; the more preferred hydrophobic acrylic monomers are stearyl methacrylate and n-butyl acrylate.

In the preferred form of an embodiment of the present invention the anionic acrylic monomers dissolved in the aqueous phase are a mixture of at least one monomer containing a strongly acidic functional group (AF) and one or more monomers containing a carboxylic group (AC), the weight ratio between AF and AC being comprised from 4:1 and 1:1, more preferably from 2.5:1 and 1.5:1.

In another preferred embodiment of the present invention the anionic acrylic monomers dissolved in the aqueous phase are a mixture of at least one monomer containing a strongly acidic functional group (AF) and one or more monomers containing a carboxylic group (AC), the weight ratio between AF and AC being higher than 4:1, more preferably from 4:1 and 20:1, AF consisting of 2-acrylamido-2-methylpropanesulfonic acid, its salts or mixture thereof.

Preferably the anionic acrylic monomers containing a carboxylic group are chosen between acrylic acid and methacrylic acid.

In still another preferred form of an embodiment of the present invention the anionic acrylic monomers dissolved in the aqueous phase consists of 2-acrylamido-2-methylpropanesulfonic acid, its salts or mixture thereof.

In the procedure of the invention, normally, the alkali used is NaOH.

According to a preferred aspect of the invention the anionic acrylic polymer obtained by inverse emulsion polymerization is cross-linked with from 0.01% to 1% by weight on the total weight of the monomers of a compound containing two or more ethylenic groups, more preferably with methylene-bis-acrylamide, Among the initiators of radical polymerization utilizable for embodiments of the present invention are ammonium, potassium or sodium persulfate, and water soluble organic peroxides, by way of example hydrogen peroxide and peracetic acid.

For the realization of the present invention it is also possible to use an initiator of radical polymerization which is soluble in the oil phase containing the hydrophobic acrylic monomer; examples of such initiators are lauroyl peroxide and benzoyl peroxide.

In the inverse emulsions of the invention the oil phase consists of mineral oils containing saturated hydrocarbons or by vegetable oils or by mixture thereof having boiling point from 150 to 300° C.

Preferably the organic phase is a C₁₃-C₁₆ iso-paraffin. The water-in-oil and the oil-in-water emulsifiers are those normally used for this purpose.

We cite among the utilizable water-in-oil emulsifiers: sorbitan monolaurate, sorbitan monopalmitate, sorbitan monostearate, sorbitan monooleate; among the utilizable oil-in-water emulsifiers we cite the linear or branched ethoxylated alcohols.

To initiate the polymerization of the acrylic monomers advantageously an aqueous solution of sodium metabisulfite is used.

The inverse emulsions of the invention may further additionally contain the common additives used in radical polymerization, by way of example sequestering agents such as sodium diethylenetriaminepentaacetate.

As it was previously observed, the inverse emulsions of the present invention are stable and allow the obtainment of stable cosmetic formulations; without giving an exhaustive explanation of the phenomenon it is supposed that the presence of hydrophobic side chains in the polymeric structure enhances the compatibility of the thickener with all the other organic compounds.

Polymers incorporating hydrophobic side chains are part of the state of the art of other categories of products, such as polymeric surfactants, which are however used for their surface-active properties and do not possess thickening properties.

In the following examples the preparation of inverse emulsions according to the invention and of some cosmetic formulations containing them is reported.

1. An inverse emulsion comprising the product of admixing an aqueous phase and an oil phase, wherein:
the weight ratio between the aqueous phase and the oil phase (aqueous phase:oil phase) is from 4:1 to 2:1,
the inverse emulsion contains from 20 to 70% percent by weight of an anionic acrylic polymer, the anionic acrylic polymer being obtained by inverse emulsion polymerization of a mixture of monomers consisting essentially of:
(i) one or more anionic acrylic monomers dissolved in the aqueous phase, and
(ii) at least a one hydrophobic acrylic monomer dissolved in the oil phase,
(iii) a crosslinker comprising a compound containing two or more ethylenic groups, and,
wherein
(a) at least one of the one or more anionic acrylic monomers contains a strongly acidic functional group, and
(b) the concentration of the at least one hydrophobic acrylic monomers is from 0.1% to 5% weight percent of the total weight of the one or more anionic acrylic monomers.

2. The inverse emulsion of claim 1 wherein the anionic acrylic monomers dissolved in the aqueous phase are a mixture of at least one monomer containing a strongly acidic functional group (AF) and one or more monomers containing a carboxylic group (AC), the weight ratio between AF and AC being higher than 4:1.

3. The inverse emulsion of claim 2 wherein AF consists essentially of a member selected from the group consisting of 2-acrylamido-2-methylpropanesulfonic acid, its salts and mixture thereof.

4. The inverse emulsion of claim 1 wherein the anionic acrylic monomers dissolved in the aqueous phase consist essentially of a member selected from the group consisting of 2-acrylamido-2-methylpropanesulfonic acid, its salts and mixture thereof.

5. The inverse emulsion of claim 1 wherein the hydrophobic monomer is stearyl methacrylate.

6. The inverse emulsion of claim 1 wherein the crosslinker consists essentially of ethylenebisacrylamide.

7. The inverse emulsion of claim 1 wherein the one or more monomers containing a carboxylic group (AC) is selected from the group consisting of acrylic acid, methacrylic acid, and combinations thereof.
